(19) United States
(12) Patent Application Publication
(10) Pub. No.: US 2008/0208349 A1
(43) Pub. Date: Aug. 28, 2008

(54) Expandable subtabar implant

(76) Inventor: Robert Graser, San Antonio, TX (US)

Correspondence Address: KAMMER BROWNING PLLC, 7700 BROADWAY, SUITE 202, SAN ANTONIO, TX, 78209, US

(21) Appl. No.: 11/710,246

(22) Filed: Feb. 23, 2007

(51) Int. Cl. A61F 2/42

(52) U.S. Cl. 623/21.18

(57) ABSTRACT

An expandable subtalar implant device suitable for addressing a flatfoot condition. The device is a sinus tarsi implant that blocks excessive motion between the talus and calcaneus bones in the foot while permitting normal motion and alignment. The device comprises a generally cylindrical metal structure having a first proximal expandable end and a second distal adjustment end. A first component of the implant forms each of the cylindrical end sections and a second component comprises a movable internal rod that serves to progressively expand the outer cylinder of the implant. The internal rod is externally threaded and mates with internal threading on the cylindrical outer component. Progressive turning of the internal rod engages a number of radially arranged inclined surfaces that form the interior walls of the cylindrical shell component. Progressive engagement of the rod with these inclined surfaces forces the end of the cylindrical shell component outward; expanding the overall diameter of the implant once it has been positioned within the sinus tarsi.

### BACKGROUND OF THE INVENTION

1. Field of the Invention

The present invention relates generally to medical implant devices, especially those associated with modifying skeletal structure and motion. The present invention relates more specifically to an implant device for the correction of skeletal alignment deformities in the feet.

2. Description of the Related Art

A skeletal deformity in humans commonly referred to as “flatfoot” is an excessive pronation of the foot caused by abnormal motion between two bones of the foot. The result of this abnormality is that the inside arch of the foot becomes flattened, generally as a result of the calcaneus or heel bone turning outward. This abnormal motion of the ankle bone (talus) with respect to the calcaneus can eventually cause anatomical misalignment and therefore requires some correction.

It is also known that in the above described motion between the talus and calcaneus, a naturally occurring opening (sinus) formed between the talus and calcaneus is closed. Efforts in the past therefore to correct the abnormal flatfoot condition have focused on the insertion of a prosthetic type implant into this opening, generally referred to as the sinus tarsi. The joint involved is generally referred to as the subtalar joint and is formed by the posterior talar facet of the calcaneus and the posterior calcaneal facet of the talus.

The primary technique, therefore, to correct a flatfoot condition is to use a subtalar implant that is inserted into the sinus tarsi to reposition the talus relative to the calcaneus. Various structural configurations have been offered for such an implant, although none have been found fully satisfactory in solving the problems associated with placing the implant, maintaining it in place, and of course, having the implant function properly during normal motion of the foot. The initial critical aspect of the design of the implant is the ease with which the implant may be placed, both in terms of surgical invasiveness and proper placement or alignment. Many designs previously offered have unfortunately focused almost entirely on the ease with which the implant is placed and its proper alignment within the sinus tarsi.

A second issue with subtalar implants is the ability to maintain the implant in place and to prevent misalignment or dislodgement of the implant over time. Many structural features of implants developed in previous efforts, therefore, have been directed to what has been perceived to be the necessity of maintaining the implant fixed in place within the sinus tarsi. Additionally, although most subtalar implants proposed to date achieve the function of obstructing the space formed between the talus and the calcaneus, and thereby achieve some level of functionality, various structural configurations detract from that functionality either through the aforementioned misalignment or through pain and discomfort associated with the individual receiving the implant. Efforts in the past to provide solutions to the above mentioned issues and problems have included a number reflected in the following U.S. Patents:

U.S. Pat. No. 6,168,631 issued to Maxwell et al. on Jan. 2, 2001 entitled Subtalar Implant System and Method for Insertion and Removal. This patent describes an implant in the nature of a metal screw having a number of crossing slots formed in the threads. The screw configuration is intended to secure the implant within the joint although the resultant lack of a smooth surface can lead to irritation of the bone surfaces and the surrounding tissue. Maxwell et al. further describes the use of a coaxial guide element and a sizer for placement of the implant.

U.S. Pat. No. 4,450,591 issued to Rappaport on May 29, 1984 entitled Internal Anti-Proratory Plug Assembly and Process of Installing the Same. This patent describes a plastic cone shaped plug provided with a tie line that may be inserted into the sinus tarsi or opening of the subtalar joint to correct pes plano valgus (flatfoot). The tie line is connected around the deltoid ligament after insertion of the plug.

U.S. Pat. No. 6,136,032 issued to Viladot Perice et al. on Oct. 24, 2000 entitled Implant for Correcting Flat Foot Condition. This patent describes a subtalar implant made up of a complex arrangement of multiple elements including a cylinder with serrated external protrusions and an internal expansion cone that is directed into the wedge shaped cylinder by means of a number of threaded components.

U.S. Pat. No. 5,360,450 issued to Giannini on Nov. 1, 1994 entitled Prosthesis for the Correction of Flatfoot. This patent describes a prosthetic implant intended to be inserted into the tarsal sinus that is made up of a bioreabsorbable material and is club shaped or slightly conical in configuration. A pair of wings are forced outward to help secure the implant in place.

U.S. Pat. No. 7,033,398 issued to Graham on Apr. 25, 2006 entitled Sinus Tarsi Implant. This patent describes a device that is composed of a non-metallic polymer and is structurally the combination of a frustum of a right cine (a conical formation) and an axially extending cylinder that is cannulated and additionally threaded on its exterior surface.

U.S. Patent Application Publication No. US 2005/0177165 filed by Zang et al. and published on Aug. 11, 2005 entitled Conical, Threaded Subtalar Implant. This patent application describes a subtalar implant generally conical in configuration and including a plurality of threads formed around the exterior surface of the body of the implant in order to help secure the implant in place. Specific structural geometries for the threading and the angle of the cone exterior surfaces are described.

U.S. Patent Application Publication No. US 2005/0177243 filed by Lepow et al. and published on Aug. 11, 2005 entitled Subtalar Implant Assembly. This application describes a subtalar implant that includes a rounded end cap region as well as a threaded region on the basic cylindrical implant structure. The threads have shapes that vary according to their position on the implant between the rounded end cap and the wider external face of the implant. A number of apertures are provided that extend from the external walls of the implant interior to the core.

U.S. Patent Application Publication No. US 2006/0041315 filed by Katz et al. and published on Feb. 23, 2006 entitled Subtalar Implant. This application describes an implant for insertion into the tarsal sinus that includes a metal body with a plurality of threads on one section of the body as well as a generally smooth non-threaded portion intended to make contact with the articulating surface of the bones of the joint. The configuration is intended to mimic the shape of the tarsal canal when the foot is bearing weight in an effort to distribute that weight over a relatively large surface area.

U.S. Patent Application Publication No. US 2005/0251264 filed by Katz et al. and published on Nov. 10, 2005 entitled Subtalar Implant. This application further describes the generally cylindrical metal implant having a threaded section and a separate smooth polymeric section that is designed to be positioned between the articulating bones of the joint. A pin axially disposed through the polymeric section provides the manner of attachment between the polymeric section and the metal section and thereby facilitates the rotation of the implant and engagement of the threads within the joint.

While as indicated above, many efforts have been made in the past to address the primary issues associated with insertion, placement, maintenance, comfort, and function, none of the prior efforts have managed to optimally address each of these issues. There remains a need, therefore, for a subtalar implant that is easy to insert and position within the sinus tarsi, that can be maintained in position during use, and that provides both the comfort and non-damaging effects that many of the prior efforts have failed to provide. It would be desirable if the surgical procedure for placing the implant were straightforward and simple and further allowed for easy adjustment of the implant at the time of its placement or subsequent as necessary. It would therefore be desirable if the implant device had a smooth outer surface so as to prevent discomfort to the individual and to further prevent damage to the bone surfaces and surrounding tissue during both placement and use of the device. It would be desirable if despite the smooth outer surface of the implant it remained fixed in place without becoming misaligned or slipping out from its placement during use.

### SUMMARY OF THE INVENTION

In fulfillment of the above and further objectives, the present invention provides an improved expandable subtalar implant device suitable for addressing a flatfoot condition. The device is a sinus tarsi implant that serves to block excessive motion between the talus and calcaneus bones in the foot while still permitting normal motion and alignment. The device comprises a generally cylindrical metal structure having a first cylindrical (proximal) expandable end and a second slightly tapered (distal) adjustment end. A first outer component of the implant forms the external cylindrical end sections mentioned above and a second inner component comprises a movable (rotatable) rod that serves to expand a portion of the outer cylinder component of the implant. The internal rod component is externally threaded and mates with internal threading on the cylindrical outer component. Progressive turning of the internal rod engages a number of radially arranged inclined surfaces that form the interior walls of one end of the cylindrical shell component. Progressive engagement of the rod with these inclined surfaces forces the end of the outer cylindrical component outward, expanding the overall diameter of the implant once it has been placed into position within the sinus tarsi. Basic tools may be provided to assist with the placement and/or removal of the implant.

### BRIEF DESCRIPTION OF THE DRAWINGS

FIG. 1 is a perspective view of the subtalar implant device of the present invention in its fully assembled but unexpanded condition.

FIG. 2A is an end view of the adjustment end section of the subtalar implant device of the present invention.

FIG. 2B is a partial sectional side view of the subtalar implant device of the present invention showing the internal structures of the device in dashed outline form.

FIG. 2C is an end view of the expandable end section of the subtalar implant of the present invention.

FIG. 3A is a side view of the skeletal components of a foot showing placement of the implant device of the present invention.

FIG. 3B is a top view of the skeletal components of a foot showing placement of the implant device of the present invention.

### DETAILED DESCRIPTION OF THE PREFERRED EMBODIMENTS

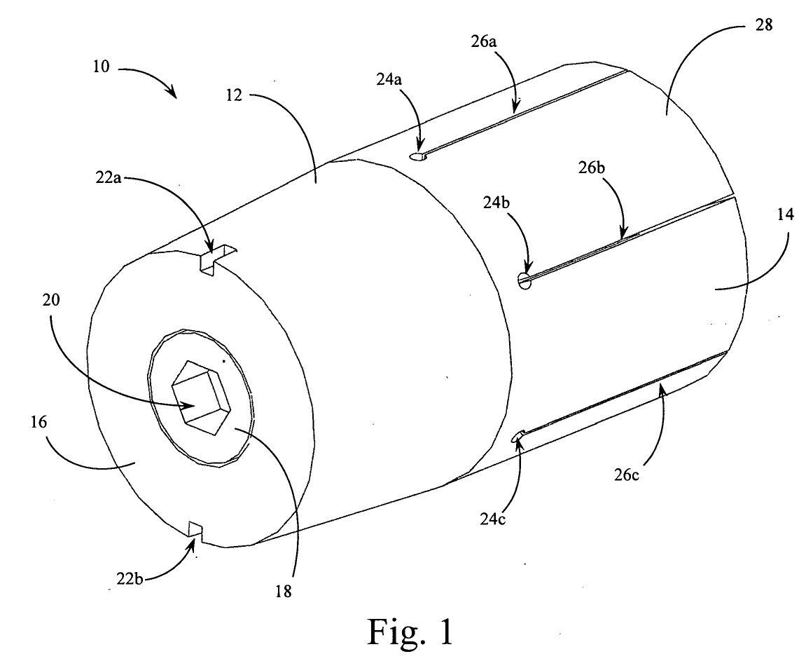

Reference is made first to FIG. 1 which discloses the overall structure of the subtalar implant device of the present invention. In FIG. 1, subtalar implant device 10 is seen in a perspective view which exhibits each of the components of the device as well as its external structural features. The tapered head section 12 of implant device 10 is generally cylindrical in structure and tapers from an approximate midpoint on the implant to what ends up being the exposed distal face 16 of the implant device. The balance of subtalar implant device 10 comprises the expansion cylinder section 14 of the device described in more detail below.

Hex screw 18 is centrally positioned within tapered head section 12 of the implant device, coaxially with the cylindrical structure of the implant device 10. As described in more detail below, hex screw 18 may, by way of engagement of a tool with internal hex socket 20, be threaded into, or threaded out from, engagement with tapered head section 12 of implant 10. The process of turning hex screw 18 within implant device 10 comprises use of a hex drive tool device (not shown) sized to appropriately engage internal hex socket 20. At the same time the hex head tool device engages hex screw 18 a separate tool, or a separate portion of a unitary tool, oppositely engages grip notches 22a and 22b. In this manner the external cylindrical structure of implant device 10 may be held stationary while hex screw 18 is turned to fix the implant device in place, again as described in more detail below.

Expansion cylinder section 14 of implant device 10 comprises a cylindrical structure divided into a number of radial segments 28. These segments 28 comprise six segments in a preferred embodiment shown in FIG. 1. Segments 28 are established by a number of flex holes drilled radially into the cylindrical walls of expansion cylinder 14, shown in FIG. 1 by flex holes 24a, 24b and 24c as examples. Joined to each of these flex holes 24a, 24b and 24c are flex slots 26a, 26b and 26c that extend from a proximal end of expandable cylinder section 14 of implant device 10 (the end not seen in the view of FIG. 1) through the walls of expansion cylinder 14 to flex holes 24a, 24b and 24c respectively. This manner of drilling apertures and cutting slots in the walls of expansion cylinder 14 creates the radially arranged expansion head sections 28 that expand outward under the influence of the internal rod to expand the overall diameter of the implant device and thereby facilitate the retention of the device in position within the sinus tarsi once the implant has been surgically positioned and placed.

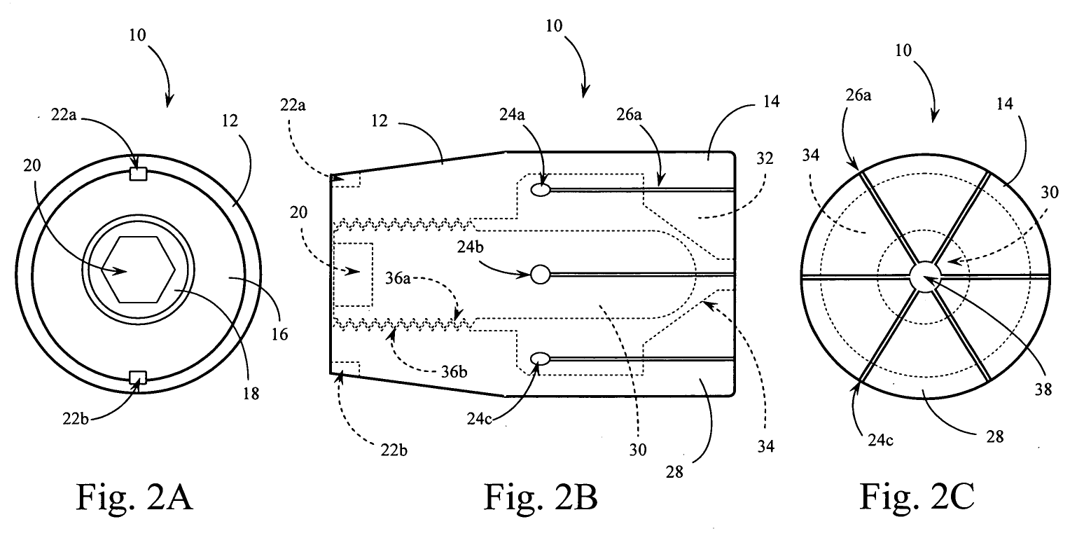

Reference is now made to FIGS. 2A through 2C for more detailed description of the structural elements of subtalar implant device 10 and the manner in which the internal threaded rod serves to expand the expansion cylinder as described above. FIG. 2A is an end view of implant device 10 showing in greater detail the tapered head section 12 of implant device 10. The slight taper extends from the overall diameter of implant device 10 (the larger external circle shown) down to the diameter of exterior (distal) face 16 (the smaller circle shown in FIG. 2A). Grip notches 22a and 22b are shown positioned at opposing points on the perimeter of the edge of exterior (distal) face 16. Internal hex socket 20 is shown positioned centrally in the exterior face of hex screw 18 which is itself positioned centrally within the exterior face 16 of subtalar implant device 10.

In FIG. 2B the internal structures of subtalar implant device 10 are disclosed in greater detail. The depth dimension of grip notches 22a and 22b is shown in dashed outline form on the top and bottom edge of the perimeter of exterior face 16 of tapered head 12. Expansion push rod 30 is shown as an extension of hex screw 18. External threads 36a on hex screw 18 are shown to engage with internal threads 36b on the interior wall of tapered head 12 of implant device 10.

The balance of the interior structures of subtalar implant device 10 are shown within the interior configuration of expansion cylinder 14. Extending from the threaded section of the interior wall described above, the otherwise solid construction of expansion cylinder 14 is hollowed out to form a number of different regions in the walls of the expansion cylinder 14. A first region extends from the area of threads 36b on the interior wall of tapered head 12, generally to a point where flex holes 24a, 24b and 24c (as examples) have been drilled through the walls of expansion cylinder 14. A relatively thin-walled portion of expansion cylinder 14 then extends somewhat more than half-way along its length toward the opposing end of expansion cylinder 14. At approximately the midpoint along the length of expansion cylinder 14 the interior walls increase in thickness and continue to increase forming inclined internal surfaces 34 to a point near the end of expansion cylinder 14 where the walls stop increasing in thickness to form the wall of aperture 38 that finally extends out from the end of expansion cylinder 14.

This internal cylindrical structure, combined with the establishment of the radially arranged expansion head sections 28, allows expansion push rod 30, under the influence of the hex tool, to be threaded into the cylindrical implant body and engage the inclined surfaces of expansion head faces 34 that form one side of expansion head wedges 32. Forcing these wedges outward results in the bending of the thin-walled sections of expansion cylinder 14 as described above. This results in an overall expansion of the external diameter of expansion cylinder 14 and therefore of the subtalar implant device 10 as a whole.

FIG. 2C is a detailed view of the opposing end of the implant device 10 from that shown in FIG. 2A. In this view expansion head sections 28 are more clearly seen as is the interior end aperture 38. The end of expansion push rod 30 is shown in dotted outline form (although it does appear through end aperture 38). The thickness of the thin-walled sections of expansion cylinder 14 is also show in dotted outline form in FIG. 2C, extending from the beginning of the thin-walled section to the centrally positioned interior end aperture 38. The additional flex slots (not numbered in this view) are likewise radially arranged with flex slots 26a, 26b, and 26c to divide expansion cylinder 14 into the six equally sized and radially arranged expansion head sections 28.

Those skilled in the art will recognize that the number and placement of the expansion sections shown in FIGS. 2A through 2C may be varied and still implement the basic concept of the present invention. Varying the number of sections along with the thickness of the bendable wall portions of the expansion cylinder will provide implant devices that are more or less easy to expand, thereby providing more or less secure placement. Implementation of the basic concepts of the present invention would generally require at least four expansion head sections be established and as many as twelve. The six expansion head sections shown in FIGS. 2A through 2C provide an optimal configuration that balances strength with flexibility in allowing for the expansion of the implant device without significantly degrading its stability once in place.

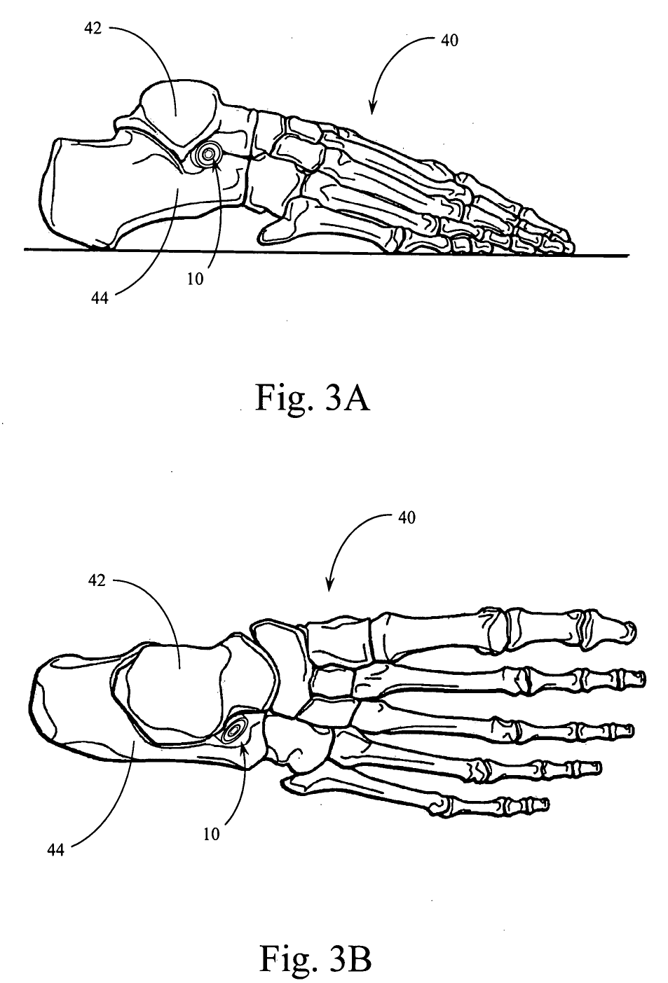

Reference is now made to FIGS. 3A and 3B for a more detailed description of the actual placement of the implant device within the skeletal structure of the foot. FIG. 3A is a side view of the skeletal structure of the foot while FIG. 3B is a top view of the same. Bones of the foot 40 in each case are shown as they are typically arranged in conjunction with the right foot of an individual. The view in FIG. 3A therefore is that of the outside portion of the right foot of an individual while the view in FIG. 3B is, as indicated, a top view of the right foot.

This orientation positions the sinus tarsi approximately one-third of the way along the length of the foot measured from the heel oriented to the front and outside portion of the foot. Implant device 10 is shown generally placed within the sinus tarsi as described above. This placement is between the talus bone 42 and the calcaneus bone 44 and generally serves to modify the motion of these two bones one against the other. The view in FIG. 3A provides perhaps the best indication of the manner in which the flatfoot condition is prevented by maintaining the “elevated” distance between the talus bone 42 and the calcaneus bone 44. Absent placement of the subtalar implant device 10 the bones described could collapse to close the tarsal sinus normally defined between these bones.

Placement of the implant device 10 initially follows a fairly well established surgical procedure for implanting any of the prior art subtalar implant devices in terms of incisions, positioning of the device, and orientation of the same. Once in position, however, the unexpanded device of the present invention is then set in place by the use of a pair of tools (or two components of a single tool) positioned on the exterior or distal face of the implant as described above. A first tool element serves to hold and prevent the rotation of the external cylindrical component of the implanted device, while a second tool element (a hex key) serves to turn or rotate the internal expansion rod component of the implant in a manner that expands the outer expansion cylinder, again as described above. Therefore, after an incision has been made and the device has been positioned and placed within the sinus tarsi, the tools are placed on the distal end face that remains exposed in the manner described. The internal push rod is rotated which advances the rod proximally into the implant device and expands the exterior expansion cylinder to increase the diameter and effectively retain the device within the cavity defined by the sinus tarsi.

Adjustment of the device once placed as described above may involve slightly loosening the hex screw, thereby slightly decreasing the expansion cylinder diameter to allow for the repositioning or realignment of the implant device as necessary. Such action could also be carried out at a point subsequent in time after surgery with relatively little invasion, again by slightly loosening the expansion cylinder and then retightening or reexpanding it when the preferred position and alignment has been established.

A number of common tools or specifically designed tools may be used for positioning, orientation and placement of the implant device of the present invention. The use of a hex socket to drive the turning of the threaded push rod in the implant device in order to initiate the expansion of the device may in part facilitate the orientation of the implant device once it is positioned in the sinus tarsi. A hex drive tool positioned on the end of a screwdriver or the like may allow the physician to orient the device and position it appropriately by manipulating the handle of the tool. Thereafter, once the external cylinder is fixed in place, the screwdriver handle of the hex drive tool may be turned to expand the implant and set it in place. In the simplest form, a hex drive screwdriver in combination with right angled needlenose pliers are all that is required to position, orient and set the implant device in place. The present invention envisions, however, the use of a specialized tool that incorporates both the hex drive component, a grasping component, and a pair of fixed plier tips into a single tool sized to fit directly over the top of the distal end of the implant device as it is manipulated by the physician. Any of these various tool embodiments would be appropriate for use in conjunction with the basic structure of the implant device of the present invention.

In addition to the above described tools that may be used to facilitate the process of fixing the implant in place and/or loosening the implant for removal, a simple threaded rod tool may be utilized to facilitate the process of placing the implant, and perhaps more importantly, the process of removing the implant. Hex screw 18 (as shown in FIG. 2B, for example) may be removed entirely from tapered head section 12 of the implant device 10 to allow access to the internal threading 36b on the interior wall of tapered head 12. A tool constructed simply of a rod with a threaded end and a hand grip opposite the threaded end, may be used to hold the implant device 10 for either placement or removal. Partially threading the threaded rod tool into the place of the hex screw 18 in the implant provides a stable means for manipulating the implant by hand. For placement of the implant, the threaded rod tool would be removed once the implant is in position and the hex screw 18 (retained loosely on the end of a hex head tool) may be threaded into place. For removal of the implant, the hex screw 18 may be removed from the implant device 10 using a hex head tool and the threaded rod tool may be threaded into its place. Gentle retraction force is then all that is necessary to remove the implant, even an implant that has been in place for some time.

Although the present invention has been described in terms of the foregoing preferred embodiments, this description has been provided by way of explanation only, and is not intended to be construed as a limitation of the invention. Those skilled in the art will recognize modifications of the present invention that might accommodate specific patients and skeletal structures. As is known in the art, it is necessary to provide various sizes of a similarly structured implant device in order to accommodate patients of different ages and different skeletal structures. Such modifications as to components, size, and even configuration where such modifications are merely coincidental to the size of the patient, do no necessarily depart from the spirit and scope of the invention. It is further anticipated that some variation may occur, for example, in the configuration and number of the expansion head sections of the implant device to allow variations in the expansion force required and experienced when placing the device. Again, all of these various modifications and variations do not necessarily depart from the spirit and scope of the invention.

I claim:

1. A subtalar implant device to facilitate the treatment of a flatfoot condition, the implant device comprising:
an external cylindrical shell component, the cylindrical shell component comprising an adjustment end section and an expansion end section, the expansion end section having angled interior walls; and
an internal expansion push rod component coaxially positioned within the cylindrical shell component and threaded therewith, the push rod component engaging the angled interior walls of the cylindrical shell component and expanding the walls outward with lateral motion of the push rod component.

2. The implant device of claim 1 wherein the cylindrical shell component and the expansion push rod component each comprise stainless steel.

3. The implant device of claim 1 wherein the expansion end section of the cylindrical shell component further comprises a plurality of expansion segments, each of the expansion segments having a bendable thin-wall root section, a rigid wedge mid-section defining one of the angled interior walls, and an external face end section.

4. The implant device of claim 3 wherein the expansion segments each comprise radially arrayed walls defining a plurality of slots extending along a long axis of the cylindrical shell component from a distal face of the expansion end section to a plurality of orthogonal apertures positioned generally at a juncture between the expansion end section and the adjustment end section of the cylindrical shell component, wherein the plurality of apertures facilitate the bending of the bendable thin-wall root sections of the expansion segments.

5. The implant device of claim 3 wherein the plurality of expansion segments comprise from four to eight segments inclusive, each of the expansion segments comprising approximately equal radial portions of the expansion end section of the cylindrical shell component.

6. The implant device of claim 5 wherein the plurality of expansion segments comprise six segments, each of the expansion segments comprising approximately 60° of the expansion end section of the cylindrical shell component.

7. The implant device of claim 1 wherein the adjustment end section of the cylindrical shell component further comprises an externally tapered outer wall and an internally threaded inner wall, the internally threaded inner wall for threaded reception of the expansion push rod component.

8. The implant device of claim 1 wherein the adjustment end section of the cylindrical shell component further comprises at least one edge slot for receiving a tool to prevent rotation of the cylindrical shell component upon directed rotation of the expansion push rod component.

9. The implant device of claim 1 wherein the expansion push rod component comprises a generally cylindrical rod having a threaded end section and an opposing hemispherical end section, the threaded end section engaging the adjustment end section of the cylindrical shell component and the hemispherical end section positioned to make contact with the angled interior walls of the expansion end section of the cylindrical shell component.

10. The implant device of claim 9 wherein the threaded end section further comprises a screw head for receiving a tool suitable for turning the expansion push rod component, wherein the turning of the push rod component directs the hemispherical end section against the angled interior walls and causes the expansion of the expansion end section of the cylindrical shell component.

11. The implant device of claim 10 wherein the screw head of the threaded end section of the expansion push rod component comprises a hex socket for receiving a hex head tool suitable for turning the push rod component.

12. A subtalar implant device to facilitate the treatment of a flatfoot condition, the implant device comprising:
an external cylindrical shell component having an adjustment end section and an expansion end section, the expansion end section having a plurality of expansion segments, each of the expansion segments having a bendable thin-wall root section, a rigid wedge mid-section defining an angled interior wall, and an external face end section, the adjustment end section comprising an externally tapered outer wall and an internally threaded inner wall; and
an internal expansion push rod component coaxially positioned within the cylindrical shell component and threaded therewith, the push rod component engaging the angled interior walls of the shell component and expanding the walls outward with lateral motion thereof, the expansion push rod component comprising a generally cylindrical rod having a threaded end section and an opposing hemispherical end section, the threaded end section engaging the adjustment end section of the cylindrical shell component and the hemispherical end section positioned to make contact with the angled interior walls of the expansion end section of the cylindrical shell component, the threaded end section further comprises a screw head for receiving a tool suitable for turning the expansion push rod component; wherein the turning of the push rod component directs the hemispherical end section thereof against the angled interior walls of the expansion segments and causes the expansion of the expansion end section of the cylindrical shell component.

13. A method for the placement a subtalar implant device into a patient, the implant device provided to facilitate the treatment of a flatfoot condition in the patient, the method comprising the steps of:
providing a subtalar implant device having an expandable cylindrical shell component and an internal expansion push rod component removably and coaxially positioned within the expandable cylindrical shell component and progressively engageable therewith to force the expansion of the expandable cylindrical shell component;
providing a first hand tool having a working end configured for directing the progressive engagement of the internal expansion push rod in the implant device;
providing a second hand tool having a working end configured to be coaxially positioned within the expandable cylindrical shell component of the implant device in place of the internal expansion push rod;
separating the internal expansion push rod component from the expandable cylindrical shell component;
positioning the working end of the second hand tool within the expandable cylindrical shell component of the implant device;
placing the implant device into the patient with the second hand tool;
removing the second hand tool from the implant device;
engaging the working end of the first hand tool with the internal expansion push rod;
directing the progressive engagement of the internal expansion push rod in the implant device to expand the expandable cylindrical shell component and thereby set the implant device within the patient; and
removing the first hand tool from the internal expansion push rod.

14. A method for the removal of a subtalar implant device from a patient, the implant device having been provided to facilitate the treatment of a flatfoot condition in the patient, the method comprising the steps of:
providing a subtalar implant device having an expandable cylindrical shell component and an internal expansion push rod component removably and coaxially positioned within the expandable cylindrical shell component and progressively engageable therewith to force the expansion of the expandable cylindrical shell component;
providing a first hand tool having a working end configured for directing the progressive disengagement of the internal expansion push rod from the implant device;
providing a second hand tool having a working end configured to be coaxially positioned within the expandable cylindrical shell component of the implant device in place of the internal expansion push rod;
engaging the working end of the first hand tool with the internal expansion push rod;
directing the progressive disengagement of the internal expansion push rod from the implant device to contract the expandable cylindrical shell component and thereby loosen the implant device within the patient;
separating the internal expansion push rod component from the expandable cylindrical shell component;
positioning the working end of the second hand tool within the expandable cylindrical shell component of the implant device;
removing the loosened implant device from the patient with the second hand tool; and
removing the second hand tool from the implant device.
